(19) United States
(12) Patent Application Publication
(10) Pub. No.: US 2025/0213438 A1
(43) Pub. Date: Jul. 3, 2025

(54) BENTON GEL ACTIVATOR COMPOSITION

(71) Applicant: SHISEIDO COMPANY, LTD., Tokyo (JP)

(72) Inventors: Satoshi YAMAKI, Yokohama (JP); Lethu NGUYEN, East Windsor, NJ (US); Alicja MIKULKO, East Windsor, NJ (US)

(73) Assignee: SHISEIDO COMPANY, LTD., Tokyo (JP)

(21) Appl. No.: 18/851,876

(22) PCT Filed: Mar. 28, 2023
(86) PCT No.: PCT/IB2023/053096

§ 371 Date: Sep. 27, 2024

Related U.S. Application Data
(60) Provisional application No. 63/325,002, filed on Mar. 29, 2022.

(51) Int. Cl. A61K 8/06; A61Q 1/02; A61Q 17/04; A61Q 19/00

(52) U.S. Cl. CPC A61K 8/064; A61Q 1/02; A61Q 17/04; A61Q 19/007; A61K 2800/30

(57) ABSTRACT

The present application provide a cosmetic composition that includes (a) a modified hectorite organoclay; (b) at least one hydrophobic solvent; and (c) at least one amphiphilic agent.

### CROSS-REFERENCE TO RELATED APPLICATIONS

The present application claims priority under 35 U.S.C. § 371 to International Patent Application No. PCT/IB2023/053096, filed on Mar. 28, 2023, which claims priority to U.S. provisional application No. 63/325,002, filed on Mar. 29, 2022. The contents of these applications are hereby incorporated by reference in their entireties.

### FIELD

The present disclosure generally relates to cosmetic compositions and more specifically to cosmetic compositions, which may not contain certain ingredients, such as a polyethylene glycol based surfactant, and their methods of making and methods of use.

### SUMMARY

One embodiment is a cosmetic composition comprising: (a) a modified hectorite organoclay; (b) at least one hydrophobic solvent; and (c) at least one amphiphilic agent.

Another embodiment is a cosmetic method comprising applying such cosmetic composition to a keratinous surface.

### FIGURES

FIGS. 1A-1C show plots with viscosity in mPa-s versus a concentration of bentone gel activator for FIG. 1A phenoxyethanol; FIG. 1B methylhepthylglycerin and FIG. 1C propanediol.

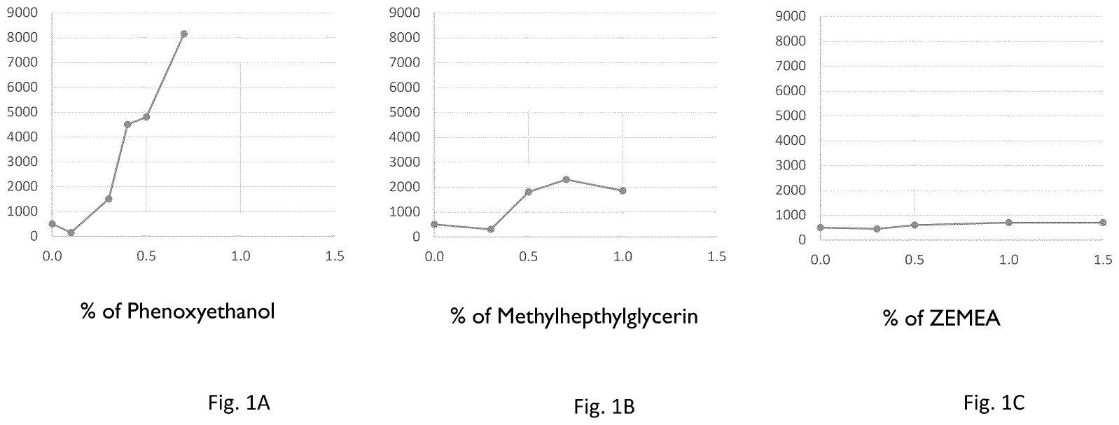

### DETAILED DESCRIPTION

Unless otherwise specified “a” or “an” means one or more.

As used herein, the term “about” placed before a specific numeric value may mean ±20% of the numeric value; ±18% of the numeric value, ±15% of the numeric value; ±12% of the numeric value; ±8% of the numeric value; ±5% of the numeric value; ±3% of the numeric value; ±2% of the numeric value; ±1% of the numeric value or ±0.5% of the numeric value.

All content information for ingredients of compositions expressed as percent (%) refers to percent (%) by mass, relative to the total mass of the composition, unless specified otherwise.

As used herein, the expression “water-in-oil composition” and related expressions refers to an water-in-oil type emulsified composition in which an aqueous (water) phase is dispersed in an oil phase. As used herein, the expression “microplastic beads” refers to particles containing a solid polymer, to which additives or other substances may have been added, and where ≥1% w/w of particles have (i) all dimensions 0.1 μm≤x≤5 mm, or (ii), for fibres, a length of 0.3 μm≤x≤15 mm and length to diameter ratio of >3. The expression “particles containing a solid polymer” refers either (i) a particle of any composition with a continuous solid polymer surface coating of any thickness or (ii) particles of any composition with a solid polymer content of ≥1% w/w.

Impurities in polyethylene glycol (PEG) based surfactants, such as 1,4 dioxane and ethylene oxide may present concerns for satisfying “clean beauty” requirements, such as safety, non-toxicity and transparency in labeling, for cosmetic products Also many consumers have an environmental concern about a low biodegradability of silicone-based ingredients, such as silicone solvents. However, eliminating PEG based ingredients, such as PEG surfactants, and/or silicone-based ingredients make create challenges for formulating cosmetic compositions. For example, elimination of those ingredients may affect stability of cosmetic compositions. Many water-in-oil compositions use organoclay-gel systems, such as bentonite-gel systems, with PEG based ingredients, such as PEG surfactants, and silicone solvents. Eliminating PEG surfactants and/or silicone solvents from such compositions makes it difficult to activate organoclay gel, such as bentonite gel, and to make the compositions stable.

In some embodiments, the present disclosure may provide cosmetic compositions, which are stable and which provide activation for organoclay gel, such as bentonite gel, without PEG based ingredients, such as PEG surfactants, and/or silicone ingredients, such as silicone solvents. Yet in some other embodiments, the present disclosure may provide cosmetic compositions, which may be used with a PEG surfactant(s) and/or a silicone solvent.

The present disclosure provides a cosmetic composition, which may include (a) a modified hectorite organoclay; (b) at least one hydrophobic solvent; and (c) at least one amphiphilic agent.

As used herein, the term “free of” may means that a total content in the cosmetic composition of ingredient(s) such as those described beneath means that those ingredient(s) is (are) not present at all in the composition.

The terms “activation” and “activating” may refer to formation of a gel structure from the organoclay in the composition.

In some embodiments, the present composition may be free of synthetic polymers.

In some embodiments, the present composition may be free of any polyethylene glycol containing ingredients, such as PEG surfactants, any polymeric ingredients, such as (meth) acrylates or (meth) acrylamide; and/or any silicones, such as silicone solvents.

In some embodiments, the composition may be free of any microplastic particles as defined above. Therefore, the present composition may qualify within “clean beauty” cosmetics category.

In some embodiments, the cosmetic composition may have sun protection properties. For example, the cosmetic composition may have a sun protection factor SPF at least 2 or at least 5 or at least 10 or at least 20 or at least 30 or at least 40 or at least 50 or at least 60 or at least 70 or at least 80 or at least 90 or at least 100.

The composition may be used in a number of cosmetics products. For example, the present composition may be used in a skin care product, such as a skincare cream, a sun screen product, a concealer product, a primer product; a foundation product, a hair product, a deodorant, a lip product, such as a lipstick or lip balm, a moisturizer, such as a color or tinted moisturizer.

In some embodiments, the cosmetic composition may be used alone. In other words, the composition may be applied alone, without another composition, to a keratinous surface or substrate, such as skin, e.g. lips, eyelids, face, or hair, of a subject, such as a human.

In some embodiments, the composition, which may be a cosmetic composition, may be used together with another product, for example, a top coat, a primer, or a powder.

In many embodiments, the composition may be a water-in-oil composition. Such water-in-oil composition may have a reduced oil leaking, which may be a leaking of a hydrophobic solvent. In many embodiments, the composition may have no measurable oil leaking or no measurable leaking of a hydrophobic solvent at all.

For example, in some embodiments, a gel system in a water-in-oil composition may contain from 1% to 90% of a hydrophobic solvent, such as oil. In some embodiments “oil leaking” may refer to moving away of the hydrophobic solvent(s), such as oil, from the structure of a gel, which may cause separation of phases and/or non-uniform accumulation of oil droplets over the gel structure. As the result, a composition with such “oil leaking” may become unstable over time.

In many embodiments, the present composition may have good spreadability and/or sensory properties due to its viscosity. This may mean that the composition when applied is uniform, smooth and does not contain clumps.

Water

The aqueous phase of the composition comprises water. A content of water in the composition may vary. In some embodiments, an amount of water may be from 1 mass % to 90 mass % or from 5 mass % to 80 mass % or from 10 mass % to 70 mass % or from 15 mass % to 75 mass % or any value or subrange within these ranges. In some embodiments, the composition may comprise one or more hydrophilic glycols, which may reduce water content of the composition.

### Modified Hectorite Organoclay

In some embodiments, the modified hectorite organoclay may be selected from, for example, dimethyl distearyl ammonium hectorite, dimethyl distearyl ammonium bentonite, and dimethyl distearyl ammonium modified montmorillonite and others, as described and exemplified in U.S. Pat. Pub. No. 2007/0071703, which is hereby incorporated by reference.

In some embodiments, the modified hectorite organoclay may be selected from those in which a quaternary ammonium salt compound is added to a natural or synthetic smectite clay mineral, such as bentonite, by way of an ion exchange reaction. The choice of modified hectorite organoclays is not particularly limited as long as it is cosmetically acceptable and may include, for example, dimethyl ammonium hectorite, and benzyl dimethyl stearyl ammonium hectorite.

In some embodiments, the modified hectorite organoclay may be bentonite (disteardimonium hectorite).

In some embodiments, the modified hectorite organoclay may be in a form of a powder.

In some embodiments, the modified hectorite organoclay may be Bentone® 38, which an organic derivative of a hectorite clay, in a powder form commercially available from Elementis Specialties Inc.

An amount of the modified hectorite organoclay in the composition may vary. In some embodiments, a content of the modified hectorite organoclay in a powder form, such as disteardimonium hectorite, may be from 0.01 mass % to 20 mass % or from 0.05 mass % to 10 mass % or from 0.1 mass % to 8 mass % or from 0.2 mass % to 5 mass % or from 0.5 mass % to 3 mass % or any value or subrange within these ranges.

### Solvent

In some embodiments, the at least one hydrophobic solvent may include at least one oil solvent. The term “hydrophobic solvent” may refer to a solvent that repels water because it is nonpolar.

In some embodiments, the at least one oil solvent may comprise at least one hydrocarbon oil solvent.

In some embodiments, the at least one oil solvent may comprise at least one non-polar oil solvent.

A non-polar oil solvent may one or more of non-polar oils disclosed in U.S. Pat. No. 10,154,954, which is incorporated herein by reference it its entirety.

Nonpolar oils are usually hydrocarbons. They lack an electronegative element like oxygen, which results in their typical hydrocarbon feel.

These oils may be of vegetable, mineral or synthetic origin.

The term “non-polar oil” may mean an oil for which the solubility parameter at 25 degrees centigrade, deltaₐ, as defined in U.S. Pat. No. 10,154,954 is equal to 0 (J/cm³)^{1/2}.

The term “hydrocarbon oil” may mean an oil formed essentially from, indeed even composed of, carbon and hydrogen atoms, and optionally oxygen and nitrogen atoms, and not containing any silicon or fluorine atoms. It may contain alcohol, ester, ether, carboxylic acid, amine and/or amide groups.

In some embodiments, the non-polar oil may include one or more non-volatile non-polar hydrocarbon oils.

The non-volatile non-polar hydrocarbon oil may be chosen from linear or branched hydrocarbons of mineral or synthetic origin, such as: liquid paraffin or derivatives thereof, squalane, isoeicosane, naphthalene oil, alkane; polybutylenes such as Indopol H-100 (molar mass or MW=965 g/mol), Indopol H-300 (MW=1340 g/mol) and Indopol H-1500 (MW=2160 g/mol) sold or manufactured by the company Amoco, hydrogenated polyisobutylenes such as Parleam (R) sold by the company Nippon Oil Fats Corporation, Panalane H-300 E sold or manufactured by the company Amoco (MW=1340 g/mol), Viseal 20000 sold or manufactured by the company Synteal (MW=6000 g/mol) or Rewopal PIB 1000 sold or manufactured by the company Witco (MW=1000 g/mol), decene/butene copolymers, polybutene/polyisobutene copolymers, especially Indopol L-14, polydecenes and hydrogenated polydecenes such as: Puresyn 10 (MW=723 g/mol) and Puresyn 150 (MW=9200 g/mol) sold or manufactured by the company Mobil Chemicals, and mixtures thereof.

In some embodiments, the at least one non-polar oil is chosen from hydrogenated polyisobutenes and/or polybutenes.

In some embodiments, the at least one non-polar oil may include squalane (a triterpene consisting of 2,6,10,15,19,23-Hexamethyltetracosane).

In some embodiments, the at least one non-polar oil may include C₉-C₁₄ alkanes, such as isoparaffins, isododecane and isohexadecane. In some embodiments, the at least one non-polar oil may include Vegelight C912-LC, which is C₉-₁₂ Alkanes (and) Coco-caprylate/caprate.

In some embodiments, the at least one oil solvent may comprise at least volatile oil solvent and at least one non-volatile oil solvent. In some embodiments, the at least one oil solvent may comprise a volatile hydrocarbon oil and a non-volatile hydrocarbon oil.

A volatile oil, such a volatile hydrocarbon oil, refers to an oil, such as hydrocarbon oil, having a boiling point lower than 300° C. at 1 atm. Examples of volatile hydrocarbon oils include, but not limited to, C₉-C₁₄ alkanes, such as isoparaffins, isododecane and isohexadecane. In some embodiments, a volatile hydrocarbon oil may be Vegelight C912-LC, which is C₉-₁₂ Alkanes (and) Coco-caprylate/caprate.

A non-volatile oil, such as non-volatile hydrocarbon oil, refers to an oil, such as a hydrocarbon oil, having a boiling point of 300° C. or higher at 1 atm, Examples of non-volatile hydrocarbon oils include, but not limited to, light squalane, hydrogenated polydecene and vaseline.

A content of a volatile oil, such as a volatile hydrocarbon oil, in the composition may vary. For example, in some embodiments, an amount of a volatile oil, such as a volatile hydrocarbon oil may be from 0.1 mass % to 99 mass % or from 1 mass % to 90 mass % or from 3 mass % to 50 mass % or any value or subrange within these ranges.

A content of a non-volatile oil, such as a non-volatile hydrocarbon oil, in the composition may vary. For example, in some embodiments, an amount of a non-volatile oil, such a non-volatile hydrocarbon oil, may be from 0.1 mass % to 99 mass % or from 1 mass % to 90 mass % or from 3 mass % to 50 mass % or any value or subrange within these ranges.

A mass ratio between a volatile oil, such as a volatile hydrocarbon oil, and a non-volatile oil, such as a non-volatile hydrocarbon oil, may vary. In some embodiments, a mass ratio between a volatile oil, such as a volatile hydrocarbon oil, and a non-volatile oil, such as a non-volatile hydrocarbon oil, may be from 1:10 to 10:1 or from 1:5 to 5:1 or from 1:3 to 3:1 or from 1:2 to 2:1 or any value or subrange within these ranges.

### Amphiphilic Agent

An amphiphilic agent is a compound comprising one or more hydrophilic parts that make the compound partially water-soluble and one or more hydrophobic parts via which the compound associates and/or interacts with an oily phase.

In some embodiments, the at least one amphiphilic agent may be at least one agent selected from diols, aromatic alcohols, glycerin ethers and their combinations.

In some embodiments, the at least one amphiphilic agent may include at least one liquid amphiphilic agent.

In some embodiments, the at least one amphiphilic agent may comprise at least one diol or glycol.

In some embodiments, the at least one amphiphilic agent may comprise at least one liquid glycol, such as glycerin, propanediol, butylene glycol, propylene glycol, ethylene glycol, and dipropylene glycol, which are water-soluble and partially solved to oils.

In some embodiments, an amount of the at least one liquid amphiphilic agent, such as glycerin, propanediol, butylene glycol, propylene glycol, and dipropylene glycol, may be 0.01 mass % to 10 mass % or from 0.05 mass % to 8 mass % or from 0.1 mass % to 5 mass % or from 0.3 mass % to 4 mass % or from 0.5 mass % to 3 mass % or any value or subrange within these ranges.

In some embodiments, the at least one amphiphilic agent may comprise at least one amphiphilic preservative.

In some embodiments, the composition may include at least one preservative agent. In some embodiments, the at least one preservative agent may include at least one amphiphilic liquid preservative agent, such as methylheptylglycerin and/or ethylheptylglycerin. Due to the presence of the at least one amphiphilic liquid preservative agent, such as methylheptylglycerin and/or ethylheptylglycerin, the composition may have a decreased white cast. The decrease of white cast may be achieved without using silicone(s) in the composition.

A content of the at least one amphiphilic liquid preservative agent, such as methylheptylglycerin and/or ethylheptylglycerin, in the composition may vary. In some embodiments, an amount of the at least one amphiphilic liquid preservative agent, such as methylheptylglycerin and/or ethylheptylglycerin, may be 0.01 mass % to 5 mass % or from 0.05 mass % to 4 mass % or from 0.1 mass % to 3 mass % or from 0.3 mass % to 2.5 mass % or from 0.5 mass % to 2 mass % or any value or subrange within these ranges.

In some embodiments, the at least one aromatic alcohol may include phenoxyethanol. A content of phenoxyethanol in the composition may vary. In some embodiments, an amount of phenoxyethanol may be from 0.01 mass % to 1.5 mass % or from 0.05 mass % to 1.5 mass % or from 0.05 mass % to 1.2 mass % or from 0.1 mass % to 1.2% or from 0.1 mass % to 1.0 mass % or any value or subrange within these ranges.

The at least one amphiphilic agent such as at least one liquid amphiphilic preservative, may act as a gel activator for the modified hectorite organoclay.

The at least one amphiphilic ingredient, such as at least one liquid amphiphilic preservative, may reduce a leaking of the at least one hydrophobic solvent, such as oil leaking, compared to otherwise identical compositions which do not include the at least one liquid amphiphilic ingredient, such as at least one liquid preservative. For example, the composition may have 3 months oil leaking as defined in the Examples below of no more than about 30% or no more than about 28% or no more than about 26% or any value or range with these ranges. For example, the composition may have 1 week oil leaking as defined in Examples below of a no more than about 10% or no more than about 8% or no more than about 6.5% or no more than about 6% or no more than about 5% or any value or range with these ranges.

The at least one amphiphilic agent, such as at least one liquid amphiphilic preservative, may also thicken the composition increasing the compositions viscosity. For example, the at least one amphiphilic agent, such as at least one liquid amphiphilic preservative or aromatic alcohol, may provide the composition with a viscosity of 20% higher than one without amphiphilic agent(s), 15% higher than one without amphiphilic agent(s), 10% higher than one without amphiphilic agent(s), 8% higher than one without amphiphilic agent(s), 5% higher than one without amphiphilic agent(s), 3% higher than one without amphiphilic agent(s), or 1% higher than one without amphiphilic agent(s), or any value or subrange within these ranges.

An amount of the at least one amphiphilic agent, such as at least one liquid amphiphilic preservative, may vary. In some embodiments, a content of the at least one amphiphilic agent, such as at least one liquid amphiphilic preservative, may be from 0.05mass % to 20 mass % or from 0.1 mass % to 18 mass % or from 0.2 mass % to 15 mass % or any value or subrange within these ranges. When the at least one amphiphilic agent includes phenoxyethanol, an amount of phenoxyethanol may be from 0.05 mass % to 5 mass % or from 0.1 mass % to 3 mass % or from 0.2 mass % to 1 mass % or any value or subrange within these ranges. When the at least one amphiphilic agent includes propanediol, an amount of propanediol may be from 0.05 mass % to 20 mass % or from 0.1 mass % to 15 mass % or from 0.2 mass % to 10 mass % or any value or subrange within these ranges. When the at least one amphiphilic agent includes methylhepthylglycerin, an amount of methylhepthylglycerin may be from 0.05 mass % 5 mass % or from 0.1 mass % 3 mass % or from 0.2 mass % to 2 mass % or from 0.3 mass % to 1.5 mass % or any value or subrange within these ranges.

In some embodiments, the composition may include more than one, i.e. two, three, four, etc., amphiphilic agents, such as phenoxyethanol, methylheptylglycerin, ethylhexylglycerin, caprylhydroxamic acid, propanediol, butylene glycol. For example, in some embodiments, the composition may include phenoxyethanol (0.05 mass % to 2 mass % or from 0.1 mass % to 1.8 mass % or from 0.2 mass % to 1 mass %) and one or both of propanediol (butylene alcohol (0.1 mass % to 15 mass %).

### Surfactant

In some embodiments, the composition may include at least one amphiphilic surfactant.

In some embodiments, the at least one amphiphilic surfactant may be at least one non-PEG surfactant, such as a polyglyceryl surfactant.

In some embodiments, the at least one amphiphilic surfactant may comprise at least one polyglyceryl surfactant.

At least one polyglyceryl surfactant may be selected from polyglyceryl 2 surfactants, polyglyceryl 3 surfactants, polyglyceryl 4 surfactants, polyglyceryl 5 surfactants, polyglyceryl-6 surfactants, polyglyceryl 7 surfactants, polyglyceryl 8 surfactants, polyglyceryl 9 surfactants, polyglyceryl 10 surfactants, polyglyceryl 11 surfactants, and polyglyceryl 12 surfactants.

Exemplary polyglyceryl surfactants include, but not limited to, Polyglyceryl-4 Caprate, Polyglyceryl-2 Caprate, Polyglyceryl-4 Caprylate, Polyglyceryl-6 Caprylate, Polyglyceryl-6 Caprate, Polyglyceryl-4 Caprylate/Caprate, Polyglyceryl-6 Caprylate/Caprate, Polyglyceryl-3 Cocoate, Polyglyceryl-4 Cocoate, Polyglyceryl-10 Decalinoleate, Polyglyceryl-10 Decaoleate, Polyglyceryl-10 Decacasterate, Polyglyceryl-3 Dicaprate, Polyglyceryl-3 Dicocoate, Polyglyceryl-10 Didecanoate, Polyglyceryl-2 Diisostearate, Polyglyceryl-3 Diisostearate, Polyglyceryl-10 Diisostearate, Polyglyceryl-4 Dilaurate, Polyglycerin-2 Dioleate, Polyglyceryl-3 Dioleate, Polyglyceryl-6 Dioleate, Polyglyceryl-10 Dioleate, Polyglyceryl-6 Dipalmitate, Polyglyceryl-10 Dipalmitate, Polyglyceryl-2Dipolyhydroxystearate, Polyglyceryl-2 Distearate, Polyglyceryl-3 Distearate, Polyglyceryl-6 Distearate, Polyglyceryl-10 Distearate, Polyglyceryl-10 Heptaoleate, Polyglyceryl-10 Heptastearate, Polyglyceryl-6 Hexaoleate, Polyglyceryl-10 Hexaoleate, Polyglyceryl-2 Isopalmitate, Polyglyceryl-2 Isostearate, Polyglyceryl-4 Isostearate, Polyglyceryl-5 Isostearate, Polyglyceryl-6 Isostearate, Polyglyceryl-10 Isostearate, Polyglyceryl-2 Laurate, Polyglyceryl-3 Laurate, Polyglyceryl-4 Laurate, Polyglyceryl-4 Laurate/Sebacate, Polyglyceryl-4 Laurate/Succinate, Polyglyceryl-5 Laurate, Polyglyceryl-6 Laurate, Polyglyceryl-10 Laurate, Polyglyceryl-3 Myristate, Polyglyceryl-10 Myristate, Polyglyceryl-2 Oleate, Polyglyceryl-3 Oleate, Polyglyceryl-4 Oleate, Polyglyceryl-5 Oleate, Polyglyceryl-6 Oleate, Polyglyceryl-8 Oleate, Polyglyceryl-10 Oleate, Polyglyceryl-3 Palmitate, Polyglyceryl-6 Palmitate, Polyglyceryl-10 Pentalaurate, Polyglyceryl-10 Pentalinoleate, Polyglyceryl-4 Pentaoleate, Polyglyceryl-10 Pentaoleate, Polyglyceryl-3 Pentaricinoleate, Polyglyceryl-6 Pentaricinoleate, Polyglyceryl-10 Pentaricinoleate, Polyglyceryl-4 Pentastearate, Polyglyceryl-6 Pentastearate, Polyglyceryl-10 Pentastearate, Polyglyceryl-3 Polyrisinoleate, Polyglyceryl-6 Polyricinoleate, Polyglyceryl-3 Ricinoleate, Polyglyceryl-2 Sesquiisostearate, Polyglyceryl-2 Sesquioleate, Polyglyceryl-2 Sesquistearate, Polyglyceryl-3 Stearate, Polyglyceryl-2 Stearate, Polyglyceryl-4 Stearate, Polyglyceryl-8 Stearate, Polyglyceryl-10 Stearate, Polyglyceryl-2 Tetraisostearate, Polyglyceryl-6 Tetraoleate, Polyglyceryl-10 Tetraoleate, Polyglyceryl-2 Tetrastearate, Polyglyceryl-2 Triisostearate, Polyglyceryl-3 Triisostearate, Polyglyceryl-10 Trioleate, Polyglyceryl-4 Tristearate, Polyglyceryl Tristearate, and Polyglyceryl-10 Tristearate.

In some embodiments, at least one polyglyceryl surfactants may include at least one polyglyceryl 2 surfactant, such as polyglyceryl-2 diisostearate, and/or at least one polyglyceryl 6 surfactant, such as polyglyceryl-6 polyricinoleate.

In some embodiments, polyglyceryl-6 polyricinoleate may provide the water-in-oil emulsified composition with good stability with no separation between phases and good spreadability.

In some embodiments, at least one amphiphilic surfactant may include at least one surfactant selected from polyglyceryl-2 diisostearate, polyglyceryl-6 polyricinoleate and sorbitan sesquiisostearate.

An amount of the at least one amphiphilic surfactant, such as at least one polyglyceryl surfactant, in the composition may vary. In some embodiments, the at least one amphiphilic surfactant, such as polyglyceryl surfactants may constitute from 0.1 mass % to 15 mass % or from 0.3 mass % to 15 mass % or from 0.5 mass % to 10 mass % or 1 mass % to 7 mass % or any value or subrange within these ranges.

In some embodiments, an amount of polyglyceryl-6 surfactant, such as polyglyceryl-6 polyricinoleate may be from 0.1 mass % to 5 mass % or from 0.3 mass % to 4 mass % or from 0.5 mass % to 3 mass % or any value or subrange within these ranges of the composition.

In some embodiments, an amount of polyglyceryl-2 surfactant, such as polyglyceryl-2 diisostearate may be from 0.1 mass % to 5 mass % or from 0.3 mass % to 4 mass % or from 0.5 mass % to 3 mass % or any value or subrange within these ranges of the composition.

### Pigments

In some embodiments, the composition may also include one or more pigments. In some embodiments, one or more pigments may be in a form of a powder. In some embodiments, a powder used as a pigment may be a hydrophobically surface treated powder, which may be dispersed in the oily phase of the composition.

In some embodiments, a powder used as a pigment may be a pigment grade powder. The term “pigment grade” may mean an average particle size in the powder of about 0.2 to 0.4 μm. In some embodiments, one or more pigments may be selected from pigment-grade titanium dioxide, pigment-grade zinc oxide, fine particle zinc oxide, talc, mica, sericite, kaolin, titanated mica, black iron oxide, yellow iron oxide, red iron oxide, ultramarine, Prussian blue, chromium oxide, chromium hydroxide, silica and cerium oxide. In some embodiments, one or more pigments may include one or more mineral pigments, such as one or more iron oxide pigments, such as black iron oxide, yellow iron oxide and red iron oxide. In some embodiments, one or more mineral pigments, such iron oxide pigments, may be hydrophobically surface treated with, for example, lauroyl lysine. Lauroyl lysine treated iron oxide pigments are commercially available, for example, as Unipure Yellow LC 182 LL, Unipure Red LC 381 LL and Unipure Black LC 989 LL.

A content of the one or more pigments in the composition may vary. For example, in some embodiments, an amount of one or more pigments in the composition may be from 0 mass % to 30 mass % or from 0.1 mass % to 25 mass % or from 0.1 mass % to 20 mass % or 0.2 mass % to 15 mass % or any value or subrange within these ranges. Powders

In some embodiments, the composition may include one or more powders. For example, in some embodiments, the one or more powders may be dispersed in the oily phase of the water-in-oil emulsified composition. Yet in some embodiments, the one or more powders may be dispersed in the water phase of the water-in-oil emulsified composition.

In some embodiments, one or more powders may include one or more of titanium dioxide particles, silica particle, iron oxide particles and zinc oxide particles. A hydrophobization surface treatment applied to one or more powders, such as titanium dioxide particles, silica particles, iron oxide particles and/or zinc oxide particles, may be (1) a treatment with a metallic soap consisting of a higher fatty acid and a multivalent metal, such as a divalent, e.g. magnesium, or a trivalent metal, e.g. aluminum or (2) a composite treatment with a higher fatty acid and a hydroxide of a multivalent metal, such as a divalent metal, e.g. magnesium or a trivalent metal, e.g. aluminum. The higher fatty acid may be a C8 to C24, such as C12 to C22, linear or branched carboxylic acid, e.g. stearic acid or isostearic acid. For example, in some embodiments, one or more powders, such as titanium dioxide particles, silica particles iron oxide particles and/or zinc oxide particles, may be surface treated with magnesium stearate and/or magnesium isostearate or with a composite treatment of aluminum hydroxide and stearic and/or isostearic acid.

In some embodiments, one or more powders may provide at least some of sun protection properties, such as ultraviolet protection properties, to the composition. For examples, the one or more powders may comprise one or more ultraviolet scattering powders. Examples of ultraviolet scattering powders include titanium dioxide powders, zinc oxide powders, silica powders, and composite powders, such as titanium dioxide coated mica, titanium dioxide coated bismuth oxychloride, titanium dioxide coated talc and titanium dioxide coated glass flake. Ultraviolet scattering powders may have an average particle diameter of about 25 to 100 nm. In some embodiments, one or more ultraviolet scattering powders may include titatanium dioxide powder, zinc oxide powder or a combination thereof.

Ultraviolet scattering powders may hydrophobic treated on a surface of a base material, such as zinc oxide or titanium dioxide. Examples of methods of hydrophobic treatment of surface include fluorine treatment using perfluoroalkyl phosphoric acid ester, perfluoroalcohol or the like; amino acid treatment using N-acylglutamic acid or the like; lecithin treatment; metallic soap treatment; fatty acid treatment; and alkylphosphoric acid ester treatment.

A content of ultraviolet scattering powder(s) in the composition may vary. In some embodiments, an amount of the ultraviolet scattering powder(s) may be from 0.1 mass % to 30 mass % or from 0.5 mass % to 25% or from 1 mass % to 20% mass %.

### Organic Ultraviolet Absorbers

In some embodiments, the composition may comprise one or more organic ultraviolet absorbers. In such case, the one or more organic ultraviolet absorbers may provide at least some of sun protection properties, such as ultraviolet protecting properties, to the composition.

The sunlight which reaches the surface of the earth has a fraction of UV-B radiation (280 to 320 nm) and of UV-A radiation (320 to 400 nm) which directly border the visible light region. The effect on the human skin is evident particularly in the case of UV-B radiation through sunburn.

The maximum of the erythema activity of sunlight is given as the relatively narrow range around 308 nm.

To protect against UV-B radiation, numerous compounds are known, which are, inter alia, derivatives of 3-benzylidenecamphor, of 4-aminobenzoic acid, of cinnamic acid, of salicylic acid, of benzophenone, and of 2-phenylbenzimidazole.

It is also important to have available filter substances for the range between about 320 nm and about 400 nm, the so-called UV-A region, since its rays can cause reactions in photosensitive skin. It has been found that UV-A radiation leads to damage of the elastic and collagenous fibers of connective tissue, which causes the skin to age prematurely, and that it should be regarded as the cause of numerous phototoxic and photoallergic reactions. The harmful effect of UV-B radiation can also be intensified by UV-A radiation.

UV photoprotective filters which may be used are oil-soluble organic UV-A filtering compounds and/or UV-B filtering compounds and/or water-soluble UV-A filtering compounds and/or UV-B filtering compounds.

Oil and water soluble UV filtering compounds are disclosed, for example, in U.S. patent No, 8,691,196, which is incorporated herein by reference in its entirety.

In some embodiments, one or more organic ultraviolet absorbers oil-soluble organic ultraviolet absorbers, which may be dissolved in the oil phase of the composition. Non-limiting examples of such absorbers include: PABA, PEG-25 PABA, Benzylidenecamphorsulfonic acid, Camphorbenzalkonium methosulfate, Terephthalylidenedicamphorsulfonic acid, Phenylbenzimidazolesulfonic acid, Disodium phenyldibenzimidazoletetrasulfonate, Benzophenone-4, Benzophenone-5, Benzophenone-9, or mixtures thereof.

Examples of oil-soluble ultraviolet absorbers include, but not limited to benzoic acid derivatives, salicylic acid derivatives, cinnamic acid derivatives, dibenzoylmethane derivatives, β-β-diphenyl acrylate derivatives, benzophenone derivatives, benzylidene camphor derivatives, phenylbenzimidazole derivatives, triazine derivatives, phenylbenzotriazole derivatives, anthranil derivatives, imidazoline derivatives, benzal malonate derivatives, and 4,4-diarylbutadiene derivatives.

Examples of benzoic acid derivatives include, but not limited to ethyl p-aminobenzoate (PABA), ethyl dihydroxypropyl PABA, ethylhexyl dimethyl PABA (e.g., “Escalol™. 507; ISP), glyceryl PABA, PEG-25 PABA (e.g., “Uvinul™ P25”; BASF), and diethylamino hydroxybenzoyl hexyl benzoate (e.g., “Uvinul™ A Plus”).

Examples of salicylic acid derivatives include, but not limited to homosalate (“Eusolex™ HMS”; Rona/EM Industries, Inc.), ethylhexyl salicylate (e.g., “Neo Heliopan™ OS”; Haarmann & Reimer), dipropylene glycol salicylate (e.g., “Dipsal™”; Scher), and TEA-salicylate (e.g., “Neo Heliopan™ TS”; Haarmann & Reimer).

Examples of cinnamic acid derivatives include, but not limited to octyl methoxycinnamate or ethylhexyl methoxycinnamate (e.g., “Parsol™ MCX”; Hoffmann-La Roche, Ltd.), isopropyl methoxycinnamate, isoamyl methoxycinnamate (e.g., “Neo Heliopan™E1000”; Haarmann & Reimer), cinoxate, DEA methoxycinnamate, diisopropyl methylcinnamate, glyceryl ethylhexanoate dimethoxycinnamate, and di-(2-ethylhexyl)-4′-methoxybenzal malonate.

Examples of dibenzoylmethane derivatives include, but not limited to 4-tert-butyl-4′-methoxydibenzoylmethane (e.g., “Parsol™ 1789”).

Examples of β-β-diphenyl acrylate derivatives include octocrylene (e.g., Uvinul™ N539″; BASF).

Examples of benzophenone derivatives include benzophenone-1 (e.g., “Uvinul™ 400”; BASF), benzophenone-2 (e.g., “Uvinul™ D50”; BASF), benzophenone-3 or oxybenzone (e.g., “Uvinul™ M40”; BASF), benzophenone-4 (e.g., “Uvinul™ MS40”; BASF), benzophenone-5, benzophenone-6 (e.g., “Helisorb™ 11”; Norquay Technology Inc.), benzophenone-8 (e.g., “Spectra-Sorb™ UV-24”; American Cyanamid Co.), benzophenone-9(e.g., “Uvinul™DS-49”; BASF), and benzophenone-12.

Examples of the benzylidene camphor derivatives include 3-benzylidene camphor (e.g., “Mexoryl™ SD”; Chimex), 4-methylbenzylidene camphor, benzylidene camphor sulfonic acid (e.g., “Mexoryl™ SL”; Chimex), camphor benzalkonium methosulfate (e.g., “Mexoryl™ SO”; Chimex), terephthalylidene dicamphor sulfonic acid (e.g., “Mexoryl™ SX”; Chimex), and polyacrylamide methylbenzylidene camphor (e.g., “Mexoryl™ SW”; Chimex).

Examples of phenylbenzimidazole derivatives include phenylbenzimidazole sulfonic acid (e.g., “Eusolex™ 232”; Merck KGaA), and disodium phenyl dibenzimidazole tetrasulfonate (e.g., “Neo Heliopan™ AP”; Haarmann & Reimer).

Examples of triazine derivatives include anisotriazine (e.g., “Tinosorb™ S”; Ciba Specialty Chemicals Inc.), ethylhexyl triazone (e.g., “Uvinul™ T-150”; BASF), diethylhexyl butamido triazone (e.g., “Uvasorb™ HEB”; 3V SIGMA S.p.A.), and 2,4,6-tris(diisobutyl-4′-aminobenzalmalonate)-s-triazine.

Examples of phenylbenzotriazole derivatives include drometrizole trisiloxane (e.g., “Silatrizole™Rhodia Chimie), and methylene bis-benzotriazolyl tetramethylbutylphenol (e.g., “Tinosorb™ M”; Ciba Specialty Chemicals Inc.).

Examples of anthanil derivatives include menthyl anthranilate (e.g., “Neo Heliopan™ MA”; Haarmann & Reimer).

Examples of imidazoline derivatives include ethyihexyl dimethoxybenzylidene dioxoimidazoline propionate.

Examples of the benzal malonate derivatives include polyorganosiloxane having a benzal malonate functional group (e.g., Polysilicone-15; “Parsol™ SLX”; DSM Nutrition Japan K.K.).

Examples of the 4,4-diarylbutadiene derivatives include 1,1-dicarboxy (2,2′-dimethylpropyl)-4,4-diphenylbutadiene.

A content of the one or more organic ultraviolet absorbers, such as one or more oil soluble ultraviolet absorbers, may vary. In some embodiments, the composition may contain no such absorbers at all. In such case, sun protection properties of the composition may be due to ultraviolet scattering powder(s). Yet in some embodiments, the composition may contain the one or more organic ultraviolet absorbers, such as one or more oil soluble ultraviolet absorbers, in an amount from 0.1 mass % to 30 mass % or from 0.5 mass % to 25mass % or 1 mass % to 20 mass % of the composition.

Exemplary composition

In some embodiments, the composition may include 0.5 mass % to 5 mass of disteardimonium hectorite, 5 mass % to 15 mass % of a volatile oil solvent, such as a volatile hydrocarbon oil solvent, 5 mass % to 15 mass % of a non-volatile oil solvent, such as a non-volatile hydrocarbon oil solvent, 0.1 mass % to 10.0 mass % of at least one ingredient selected from the group consisting of phenoxyethanol, methylheptylglycerin, ethylhexylglycerin caprylhydroxamic acid, propanediol, butylene glycol and combinations thereof, and 1 mass % to 7 mass % of at least one surfactant selected from polyglyceryl-2 diisostearate, polyglyceryl-6 polyricinoleate, and a combination thereof.

### Additional Ingredients

In some embodiments, the composition may include one or more additional ingredients, such as humectant, an emollient and/or a moisturizer, such as glycerin, a salt, such as sodium chloride; an emollient, such as diethylhexyl succinate. The composition may also include one or more additional ingredients selected from water thickeners, oil thickeners, stabilizer, pH modifiers, sensory modifiers, and fragrance.

Yet in some embodiments, the composition may contain the one or more the additional ingredients in an amount from 1 mass % to 25 mass % or from 3 mass % to 20 mass % or 5 mass % to 18 mass %.

### Method of Making

The composition may be prepared by the process, which may include one or more of the following operations: dispersing a modified hecrorite organoclay, such as disteardimonium hectorite, in an oil phase, which includes at least one hydrophobic solvent, such as at least one hydrocarbol oil solvent; adding to such dispersion at least one liquid amphiphilic ingredient, such as at least one liquid amphiphilic preservative as discussed above to activate a gel from the organoclay and dispersing the product; adding at least one amphiphilic surfactant, such as at least one polyglyceryl surfactant, and dispersing the product; optionally, adding optional ingredients, such as pigments, powders, such as UV scattering powders, and/or organic ultraviolet absorbers and dispersing; forming an aqueous phase and adding it to the oil phase dispersion; final emulsifying the aqueous phase and the oil phase together to form the composition.

Embodiments described herein are further illustrated by, though in no way limited to, the following working examples.

### EXAMPLE

TABLE 1
Selected oil gel compositions testing various compounds, including preservatives, such as Phenoxyethanol, Methylheptylglycerin,
Ethylhexylglycerin, CaprylhydroxamicAcid, Propaneziol, and Butylene Glycol) as Benton gel activators.

| | | Control | Control | Control | Control | Example |
|---|---|---|---|---|---|---|
| RM Name | EU INCI | 1 | 2 | 3 | 4 | 1 |
| VEGELIGHT C912-LC | C9-12 ALKANE, COCO- | 10.0 | 10.0 | 10.0 | 10.0 | 10.0 |
| (GRANT INDUSTRIES) | CAPRYLATE/CAPRATE | | | | | |
| NEOSSANCE SQUALANE | SQUALANE | 10.0 | 10.0 | 10.0 | 10.0 | 10.0 |
| BENTONE 38VCG | Disteardimonium | 2.0 | 2.0 | 2.0 | 2.0 | 2.0 |
| | Hectorite | | | | | |
| JEFFSOL ® PROPYLENE CARBONATE | Propylene Carbonate | | 0.5 | | | |
| ISOSTEARIC ACID SX | ISOSTEARIC ACID | | | 0.5 | | |
| Glycerin 99.7% USP (Cargill) | Glycerin | | | | 0.5 | |
| PHENOXETOL NF 27255613162 | PHENOXYETHANOL | | | | | 0.5 |
| ZEMEA | PROPANEDIOL | | | | | |
| Zeastat ™ | CaprylhydroxamicAcid | | | | | |
| | and Propanediol | | | | | |
| Lexgard ™Natural MHG MB | Methylheptylglycerin | | | | | |
| BRONTIDE (genomatica) | Butylene Glycol | | | | | |
| LEXGARD E | Ethylhexylglycerin | | | | | |
| WOGEL 18DV | POLYGLYCERYL-2 | 3.0 | 3.0 | 3.0 | 3.0 | 3.0 |
| | DIISOSTEARATE | | | | | |
| SY - GLYSTERCRS - 75 | Polyglyceryl-6 | | | | | |
| | Polyricinoleate | | | | | |
| Evaluation | Viscosity (#4, 12 rpm) | 400 | 4200 | 150 | 750 | 3600 |
| | Ooil leaking (% of height | 6.25% | 0.00% | 15.63% | 6.06% | 0.00% |
| | for 1 week) | | | | | |
| | | Example | Example | Example | Example | Example |
| RM Name | EU INCI | 2 | 3 | 4 | 5 | 6 |
| VEGELIGHT C912-LC | C9-12 ALKANE, COCO- | 10.0 | 10.0 | 10.0 | 10.0 | 10.0 |
| (GRANT INDUSTRIES) | CAPRYLATE/CAPRATE | | | | | |
| NEOSSANCE SQUALANE | SQUALANE | 10.0 | 10.0 | 10.0 | 10.0 | 10.0 |
| BENTONE 38VCG | Disteardimonium | 2.0 | 2.0 | 2.0 | 2.0 | 2.0 |
| | Hectorite | | | | | |
| JEFFSOL ® PROPYLENE CARBONATE | Propylene Carbonate | | | | | |
| ISOSTEARIC ACID SX | ISOSTEARIC ACID | | | | | |
| Glycerin 99.7% USP (Cargill) | Glycerin | | | | | |
| PHENOXETOL NF 27255613162 | PHENOXYETHANOL | | | | | |
| ZEMEA | PROPANEDIOL | 0.5 | | | | |
| Zeastat ™ | CaprylhydroxamicAcid | | 0.5 | | | |
| | and Propanediol | | | | | |
| Lexgard ™Natural MHG MB | Methylheptylglycerin | | | 0.5 | | |
| BRONTIDE (genomatica) | Butylene Glycol | | | | 0.5 | |
| LEXGARD E | Ethylhexylglycerin | | | | | 0.5 |
| WOGEL 18DV | POLYGLYCERYL-2 | 3.0 | 3.0 | 3.0 | 3.0 | 3.0 |
| | DIISOSTEARATE | | | | | |
| SY - GLYSTERCRS - 75 | Polyglyceryl-6 | | | | | |
| | Polyricinoleate | | | | | |
| Evaluation | Viscosity (#4, 12 rpm) | 1050 | 1450 | 1950 | 1150 | 2100 |
| | Ooil leaking (% of height | 3.13% | 3.13% | 0.00% | 3.03% | 0.00% |
| | for 1 week) | | | | | |

The compositions in Table 1 were prepared using the following manufacturing process:
1. Disperse Disteardiamonium Hectorite in oil phase with 3000 rpm; 2. Add preservative bentone gel activator or other target ingredients, and disperse it with 3000 rpm 3. Add surfactant and disperse it with 3000 rpm.

The compositions in Table 1 were evaluated for their viscosity and oil leaking.

### Evaluation

### 1. Viscosity

Measured viscosity by BLOOKFIELD DV-1 Viscometer with LV-04, 12 rpm, 1 min

2. Oil leaking %

Filled samples in 2 oz glass bottles and put it in static for 1 week. Then measured the oil leaking and total heights from the bottom. Oil leaking (% of height)=(The height of oil leaking)/(The height of total bulk)*100 white cast.

Preservatives, such as Phenoxyethanol, Methylheptylglycerin, Ethylhexylglycerin, CaprylhydroxamicAcid, Propaneziol, and Butylene Glycol, work well as Benton gel activator and led to a stable bentonite system with non-PEG/non-silicone surfactant. The data in Table 4 shows that except for benton activator, it was difficult to thicken oil gel efficiently. Propylene carbonate is well-known activator, but it has many regulatory issues, such as CMR and Proposition 65, and petrochemical origin. The preservatives, such as Phenoxyethanol, Methylheptylglycerin, Ethylhexylglycerin, CaprylhydroxamicAcid, Propaneziol, and Butylene Glycol, showed good effect to activate bentonite without strong dispersion and special equipment.

Tables 2-4 tests effects of various amounts of phenoxyethanol, propanediol or methylheptylglycerin.

The compositions of Tables 2-4 were prepared using the following manufacturing process: 1. Disperse Disteardiamonium Hectlite in oil phase with 3000 rpm; 2. Add preservative bentone gel activator (phenoxyethanol, propanediol or methylheptylglycerin) and disperse it with 3000 rpm; 3. Add surfactant and disperse it with 3000 rpm.

The compositions of Tables 5-7 were evaluated as follows.

### Evaluation

### 1. Viscosity

Measured viscosity by BLOOKFIELD DV-1 Viscometer with LV-04, 12 rpm, 1 min

2. Oil leaking %

Filled samples in 2 oz glass bottles and put it in static for 3 months. Then measured the oil leaking and total heights from the bottom. Oil leaking (% of height)=(The height of oil leaking)/(The height of total bulk)*100 white cast.

TABLE 2
Selected oil gel compositions with various amounts of phenoxyethanol.

| | | Control | Example | Example | Example | Example | Example |
|---|---|---|---|---|---|---|---|
| RM Name | EU INCI | 5 | 7 | 8 | 9 | 10 | 11 |
| VEGELIGHT C912-LC | C9-12 ALKANE, | 10.0 | 10.0 | 10.0 | 10.0 | 10.0 | 10.0 |
| (GRANT INDUSTRIES) | COCO- | | | | | | |
| | CAPRYLATE/ | | | | | | |
| | CAPRATE | | | | | | |
| NEOSSANCE | SQUALANE | 10.0 | 10.0 | 10.0 | 10.0 | 10.0 | 10.0 |
| SQUALANE | | | | | | | |
| BENTONE 38VCG | Disteardimonium | 2.0 | 2.0 | 2.0 | 2.0 | 2.0 | 2.0 |
| | Hectorite | | | | | | |
| PHENOXETOL NF | PHENOXYETHANOL | 0.0 | 0.1 | 0.3 | 0.4 | 0.5 | 0.7 |
| 27255613162 | | | | | | | |
| WOGEL 18DV | POLYGLYCERYL-2 | 3.0 | 3.0 | 3.0 | 3.0 | 3.0 | 3.0 |
| | DIISOSTEARATE | | | | | | |
| SY - GLYSTERCRS - 75 | Polyglyceryl-6 | 1.0 | 1.0 | 1.0 | 1.0 | 1.0 | 1.0 |
| | Polyricinoleate | | | | | | |
| ISOSTEARIC ACID SX | ISOSTEARIC | 0.5 | 0.5 | 0.5 | 0.5 | 0.5 | 0.5 |
| | ACID | | | | | | |
| Evaluation | Viscosity | 500 | 150 | 1500 | 4500 | 4800 | 8150 |
| | (#4, 12 rpm) | | | | | | |
| | Oil leaking (% of | 32.26% | 25.00% | 3.13% | 0.00% | 0.00% | 0.00% |
| | height for 3 | | | | | | |
| | months) | | | | | | |

The data in Table 2 shows that small amount of phenoxyethanol improved oil leaking. Larger amounts improved oil leaking more and thickened the oil gel composition

TABLE 3
Selected oil gel compositions with various amounts of propanediol.

| | | Control | Example | Example | Example | Example |
|---|---|---|---|---|---|---|
| RM Name | EU INCI | 5 | 12 | 13 | 14 | 15 |
| VEGELIGHT C912-LC | C9-12 ALKANE, | 10.0 | 10.0 | 10.0 | 10.0 | 10.0 |
| (GRANT | COCO- | | | | | |
| INDUSTRIES) | CAPRYLATE/CAPRATE | | | | | |
| NEOSSANCE | SQUALANE | 10.0 | 10.0 | 10.0 | 10.0 | 10.0 |
| SQUALANE | | | | | | |
| BENTONE 38VCG | Disteardimonium | 2.0 | 2.0 | 2.0 | 2.0 | 2.0 |
| | Hectorite | | | | | |
| ZEMEA | PROPANEDIOL | 0.0 | 0.3 | 0.5 | 1.0 | 1.5 |
| WOGEL 18DV | POLYGLYCERYL-2 | 3.0 | 3.0 | 3.0 | 3.0 | 3.0 |
| | DIISOSTEARATE | | | | | |
| SY - GLYSTERCRS - | Polyglyceryl-6 | 1.0 | 1.0 | 1.0 | 1.0 | 1.0 |
| 75 | Polyricinoleate | | | | | |
| ISOSTEARIC ACID | ISOSTEARIC | 0.5 | 0.5 | 0.5 | 0.5 | 0.5 |
| SX | ACID | | | | | |
| Evaluation | Viscosity | 500 | 450 | 600 | 700 | 700 |
| | (#4, 12 rpm) | | | | | |
| | Ooil leaking (% of | 32.26% | 25.00% | 25.81% | 22.58% | 21.88% |
| | height for 3 | | | | | |
| | months) | | | | | |

The data in Table 3 shows that a small amount of propanediol improved oil leaking. Higher amounts improved oil leaking more and thickened the oil gel, but the effect of propanediol is lower than that of phenoxyethanol, see Table 2.

TABLE 4
Selected oil gel compositions with various amounts of Methylheptylglycerin

| | | Control | Example | Example | Example | Example |
|---|---|---|---|---|---|---|
| RM Name | EU INCI | 5 | 16 | 17 | 18 | 19 |
| VEGELIGHT C912-LC | C9-12 ALKANE, COCO- | 10.0 | 10.0 | 10.0 | 10.0 | 10.0 |
| (GRANT INDUSTRIES) | CAPRYLATE/CAPRATE | | | | | |
| NEOSSANCE SQUALANE | SQUALANE | 10.0 | 10.0 | 10.0 | 10.0 | 10.0 |
| BENTONE 38VCG | Disteardimonium | 2.0 | 2.0 | 2.0 | 2.0 | 2.0 |
| | Hectorite | | | | | |
| Lexgard ™Natural MHG MB | Methylheptylglycerin | 0.0 | 0.3 | 0.5 | 0.7 | 1.0 |
| WOGEL 18DV | POLYGLYCERYL-2 | 3.0 | 3.0 | 3.0 | 3.0 | 3.0 |
| | DIISOSTEARATE | | | | | |
| SY - GLYSTERCRS - 75 | Polyglyceryl-6 | 1.0 | 1.0 | 1.0 | 1.0 | 1.0 |
| | Polyricinoleate | | | | | |
| ISOSTEARIC ACID SX | ISOSTEARIC ACID | 0.5 | 0.5 | 0.5 | 0.5 | 0.5 |
| Evaluation | Viscosity (#4, 12 rpm) | 500 | 300 | 1800 | 2300 | 1850 |
| | Ooil leaking (% of height | 32.26% | 12.90% | 0.00% | 0.00% | 0.00% |
| | for 3 months) | | | | | |

The data Table 4 shows that a small amount of Methylheptylglycerin improved oil leaking. Higher amounts improved oil leaking more and thickened the oil gel.

Tables 5-6 test effects of various solvents on oil gel compositions.

The compositions of Table 5 were prepared using the following process: 1. Disperse Disteardiamonium Hectorite in oil phase with 3000 rpm 2. Add preservative benton activator (phenoxyethanol) and disperse it with 3000 rpm.

The compositions of Table 5 were evaluated as follows

### Evaluation

### 1. Viscosity

Measured viscosity by BLOOKFIELD DV-1 Viscometer with LV-04, 12 rpm, 1 min.

TABLE 5

| | | Control | Exam- | Control | Exam- | Control | Exam- | Control | Exam- | Control |
|---|---|---|---|---|---|---|---|---|---|---|
| RM Name | EU INCI | 6 | ple 20 | 7 | ple 21 | 8 | ple 22 | 9 | ple 23 | 10 |
| ESELAN-200 | Diisopropyl | 17 | 17 | | | | | | | |
| | Sebacate | | | | | | | | | |
| NEOSSANCE | SQUALANE | | | 17 | 17 | | | | | |
| SQUALANE | | | | | | | | | | |
| VEGELIGHT | C9-12 | | | | | 17 | 17 | | | |
| C912-LC | ALKANE, | | | | | | | | | |
| (GRANT | COCO- | | | | | | | | | |
| INDUSTRIES) | CAPRYLATE/ | | | | | | | | | |
| | CAPRATE | | | | | | | | | |
| CRODAMOL | DIETHYLHEXYL | | | | | | | 17 | 17 | |
| OSU-LQ-(JP) | SUCCINATE | | | | | | | | | |
| MARUKASOL R | Isododecane | | | | | | | | | 17 |
| (RM-01198) | | | | | | | | | | |
| JOJOBA OIL | Simmondsia Chinensis | | | | | | | | | |
| | Seed Oil | | | | | | | | | |
| CLEAR VALLEY | Sunflower oil | | | | | | | | | |
| HOSUN OIL | | | | | | | | | | |
| DM-FLUID-2CS | DIMETHICONE | | | | | | | | | |
| (KF-96A-2CS) | | | | | | | | | | |
| DM-FLUID-A-6T | DIMETHICONE | | | | | | | | | |
| (KF-96A-6T) | | | | | | | | | | |
| BENTONE 38VCG | Disteardimonium | 2 | 2 | 2 | 2 | 2 | 2 | 2 | 2 | 2 |
| | Hectorite | | | | | | | | | |
| PHENOXETOL NF | PHENOXYETHANOL | | 1 | | 1 | | 1 | | 1 | |
| 27255613162 | | | | | | | | | | |
| Evaluation | Viscosity (#4, 12 rpm) | 5100 | 9650 | 0 | 18250 | 0 | 30500 | 750 | 7650 | 0 |
| | | Exam- | Control | Exam- | Control | Exam- | Control | Exam- | Control | Exam- |
| RM Name | EU INCI | ple 24 | 11 | ple 25 | 12 | ple 26 | 13 | ple 27 | 14 | ple 28 |
| ESELAN-200 | Diisopropyl | | | | | | | | | |
| | Sebacate | | | | | | | | | |
| NEOSSANCE | SQUALANE | | | | | | | | | |
| SQUALANE | | | | | | | | | | |
| VEGELIGHT | C9-12 | | | | | | | | | |
| C912-LC (GRANT | ALKANE, | | | | | | | | | |
| INDUSTRIES) | COCO- | | | | | | | | | |
| | CAPRYLATE/ | | | | | | | | | |
| | CAPRATE | | | | | | | | | |
| CRODAMOL | DIETHYLHEXYL | | | | | | | | | |
| OSU-LQ-(JP) | SUCCINATE | | | | | | | | | |
| MARUKASOL R | Isododecane | 17 | | | | | | | | |
| (RM-01198) | | | | | | | | | | |
| JOJOBA OIL | Simmondsia Chinensis | | 17 | 17 | | | | | | |
| | Seed Oil | | | | | | | | | |
| CLEAR VALLEY | Sunflower oil | | | | 17 | 17 | | | | |
| HOSUN OIL | | | | | | | | | | |
| DM-FLUID-2CS | DIMETHICONE | | | | | | 17 | 17 | | |
| (KF-96A-2CS) | | | | | | | | | | |
| DM-FLUID-A-6T | DIMETHICONE | | | | | | | | 17 | 17 |
| (KF-96A-6T) | | | | | | | | | | |
| BENTONE 38VCG | Disteardimonium | 2 | 2 | 2 | 2 | 2 | 2 | 2 | 2 | 2 |
| | Hectorite | | | | | | | | | |
| PHENOXETOL NF | PHENOXYETHANOL | 1 | | 1 | | 1 | | 1 | | 1 |
| 27255613162 | | | | | | | | | | |
| Evaluation | Viscosity (#4, 12 rpm) | 24850 | 0 | 9700 | 0 | 7200 | 0 | 1050 | 0 | 250 |

The data in Table 5 shows that phenoxyethanol as bentone gel activator significantly increased the viscosity of oil gel compositions with diverse solvents including polar ester, non-polar hydrocarbon oils, plant oils, and silicone oils.

The data in Table 5 shows phenoxyethanol as Benton activator works with diverse solvents because it can thicken oil gel compositions with a variety solvents. Phenoxyethanol may work better with hydrocarbon solvents than with silicone solvents.

The compositions of Table 6 were prepared using the following Manufacturing Process: 1. Disperse Disteardiamonium Hectorite in oil phase with 3000 rpm; 2. Add preservative benton activator and disperse it with 3000 rpm; 3. Add surfactant and disperse it with 3000 rpm

The compositions of Table 6 were evaluated as follows:

### Evaluation

### 1. Viscosity

Measured viscosity by BLOOKFIELD DV-1 Viscometer with LV-04, 12 rpm, 1 min.

TABLE 6

| | | Exam- | Exam- | Exam- | Exam- | Exam- | Exam- | Exam- | Control | Control |
|---|---|---|---|---|---|---|---|---|---|---|
| RM Name | EU INCI | ple 29 | ple 30 | ple 31 | ple 32 | ple 33 | ple 34 | ple 35 | 15 | 16 |
| ESELAN-200 | Diisopropyl Sebacate | 17 | | | | | | | | |
| NEOSSANCE | SQUALANE | | 17 | | | | | | | |
| SQUALANE | | | | | | | | | | |
| VEGELIGHT | C9-12 ALKANE, COCO- | | | 17 | | | | | | |
| C912-LC | CAPRYLATE/CAPRATE | | | | | | | | | |
| (GRANT | | | | | | | | | | |
| INDUSTRIES) | | | | | | | | | | |
| CRODAMOL | DIETHYLHEXYL | | | | 17 | | | | | |
| OSU-LQ-(JP) | SUCCINATE | | | | | | | | | |
| MARUKASOL R | Isododecane | | | | | 17 | | | | |
| (RM-01198) | | | | | | | | | | |
| JOJOBA OIL | Simmondsia Chinensis | | | | | | 17 | | | |
| | Seed Oil | | | | | | | | | |
| CLEAR VALLEY | Sunflower oil | | | | | | | 17 | | |
| HOSUN OIL | | | | | | | | | | |
| DM-FLUID-2CS | DIMETHICONE | | | | | | | | 17 | |
| (KF-96A-2CS) | | | | | | | | | | |
| DM-FLUID-A-6T | DIMETHICONE | | | | | | | | | 17 |
| (KF-96A-6T) | | | | | | | | | | |
| BENTONE | Disteardimonium | 2 | 2 | 2 | 2 | 2 | 2 | 2 | 2 | 2 |
| 38VCG | Hectorite | | | | | | | | | |
| PHENOXETOL | PHENOXYETHANOL | 1 | 1 | 1 | 1 | 1 | 1 | 1 | 1 | 1 |
| NF 27255613162 | | | | | | | | | | |
| WOGEL 18DV | POLYGLYCERYL-2 | 3 | 3 | 3 | 3 | 3 | 3 | 3 | | |
| | DIISOSTEARATE | | | | | | | | | |
| KF-6017 | Peg-10 Dimethicone | | | | | | | | 3 | 3 |
| Evaluation | Viscosity (#4, 12 rpm) | 5650 | 13900 | 10800 | 5800 | 8850 | 8800 | 7500 | 0 | 0 |

The data in Table 6 shows that with non-PEG/non-silicone surfactants, phenoxyethanol used as bentone gel activator made good oil gel compositions with high viscosity, except control examples 15-16, which used a silicone solvent with a silicone surfactant.

Tables 7-10 show that bentone gel activator, such as phenoxyethanol, works better with polyglyceryl surfactants because it stabilizes water-in-oil compositions and provides good spreadability.

The compositions of Table 7 were prepared using the following Manufacturing Process: 1. Disperse Disteardiamonium Hectorite in oil phase with 3000 rpm; 2. Add water-soluble solvent and disperse it with 3000 rpm; 3. Add serfactant and disperse it with 3000 rpm; 4. Mix water phase and add it to oil phase with emulsification with 4000 rpm. The compositions of Table 7 were evaluated as follows:

### Evaluation

### 1. Viscosity

Measured viscosity by BLOOKFIELD DV-1 Viscometer with LV-04, 12 rpm, 1 min

2. Oil leaking

Observed and evaluated the amount of oil leaking of samples at 50C for 4 weeks
+: No oil leaking
+/−: Tiny oil leaking
−: Apparent, noticeable oil leaking

### 3. Spreadability

+: More than 70% of 10 subjects felt spreading well
+/−: More than 40% of 10 subjects felt spreading well
−: Less than 40% of 10 subjects felt the spreading well

TABLE 7
Selected water-in-oil organic SPF cream compositions

| | | Control | Example | Control | Example | Example | Example | Example |
|---|---|---|---|---|---|---|---|---|
| RM Name | EU INCI | 17 | 36 | 18 | 37 | 38 | 39 | 40 |
| DEIONIZED WATER | AQUA | To 100 | To 100 | To 100 | To 100 | To 100 | To 100 | To 100 |
| VEGELIGHT C912-LC | C9-12 ALKANE, COCO- | 10.0 | 9.5 | 10.0 | 9.5 | 9.5 | 5.0 | 2.0 |
| (GRANT INDUSTRIES) | CAPRYLATE/CAPRATE | | | | | | | |
| NEOSSANCE SQUALANE | SQUALANE | 10.0 | 10.0 | 10.0 | 10.0 | 10.0 | 5.0 | 3.0 |
| BENTONE 38VCG | Disteardimonium Hectorite | 2.5 | 2.5 | 2.5 | 2.5 | 2.5 | 2.5 | 2.5 |
| PHENOXETOL NF | PHENOXYETHANOL | | 0.7 | | 0.7 | | 0.7 | 0.7 |
| 27255613162 (in oil phase) | | | | | | | | |
| Lexgard ™Natural MHG MB | Methylheptylglycerin | | | | | 0.7 | | |
| WOGEL 18DV | POLYGLYCERYL-2 | 3.0 | 3.0 | 3.0 | 3.0 | 3.0 | 3.0 | 3.0 |
| | DIISOSTEARATE | | | | | | | |
| SY - GLYSTERCRS - 75 | Polyglyceryl-6 Polyricinoleate | | | 1.0 | 1.0 | 1.0 | 1.0 | 1.0 |
| ISOSTEARIC ACID SX | ISOSTEARIC ACID | 0.5 | 0.5 | 0.5 | 0.5 | 0.5 | 0.5 | 0.5 |
| BOTANESSENTIAL | ETHYLHEXYL OLIVATE, | 5.0 | 5.0 | 5.0 | 5.0 | 5.0 | 5.0 | 3.0 |
| OLIVE S-EHO | SQUALANE | | | | | | | |
| NEO HELIOPAN OS | ETHYLHEXYL SALICYLATE | 5.0 | 5.0 | 5.0 | 5.0 | 5.0 | 5.0 | 5.0 |
| OCTISLTE | | | | | | | | |
| PARSOL ® 340 | OCTOCRYLENE | 4.0 | 4.0 | 4.0 | 4.0 | 4.0 | 4.0 | 4.0 |
| PARSOL ® 1789 | BUTYL | 2.0 | 2.0 | 2.0 | 2.0 | 2.0 | 2.0 | 2.0 |
| | METHOXYDIBENZOYLMETHANE/ | | | | | | | |
| | AVOBENZONE | | | | | | | |
| CHIFFONSIL P-3R | Silica | 5.0 | 5.0 | 5.0 | 5.0 | 5.0 | 5.0 | 2.0 |
| (PRESPERSE | | | | | | | | |
| CORPORATION) | | | | | | | | |
| EDTA 3•NA2•H20 | Trisodium edta | 0.1 | 0.1 | 0.1 | 0.1 | 0.1 | 0.1 | 0.1 |
| STERLING USP SALT | SODIUM CHLORIDE | 0.1 | 0.1 | 0.1 | 0.1 | 0.1 | 0.1 | 0.1 |
| ZEMEA | PROPANEDIOL | 10 | 10 | 10 | 10 | 10 | 10 | 10 |
| PHENOXETOL NF 272556 | PHENOXYETHANOL | 0.3 | 0.3 | 0.3 | 0.3 | 0.3 | 0.3 | 0.3 |
| (in water phase) | | | | | | | | |
| Evaluation | Viscosity (#4, 12 rpm) | 19350 | 19700 | 5200 | 4500 | 3550 | 23050 | 50000 |
| | Oil leaking (50 C. 4 weeks) | − | +/− | − | + | + | + | + |
| | Spreadability | +/− | +/− | + | + | + | + | +/− |

The data in Table 7 shows that in the W/O cream compositions phenoxyethanol as a bentone gel activator improved oil leaking. With Polyglyceryl-6 Polyricinoleate, the stability got better from low to high viscosity. The spreadability was also improved by Polyglyceryl-6 Polyricinoleate. Phenoxyethanol and/or Methylheptylglycerin worked well as the activator.

The compositions of Table 8 were prepared using the following manufacturing process: Manufacturing Process: 1. Disperse Disteardiamonium Hectorite in oil phase with 3000 rpm; 2. Add preservative bentone gel activator and disperse it with 3000 rpm; 3. Add surfactant and disperse it with 3000 rpm; 4. Add powders and inorganic UV filters and disperse it with 4000 rpm; 5. Mix water phase and add it to oil phase with emulsification with 4000 rpm

The compositions of Table 8 were evaluates as follows:

### Evaluation:

### 1. Viscosity

Measured viscosity by BLOOKFIELD DV-1 Viscometer with LV-04, 12 rpm, 1 min

2. Oil leaking

Observed and evaluated the amount of oil leaking of samples at 50C for 4 weeks
+: No oil leaking
+/−: Tiny oil leaking
−: Apparent, noticeable oil leaking
   3. spreadability
+: More than 70% of 10 subjects felt spreading well
+/−: More than 40% of 10 subjects felt spreading well
−: Less than 40% of 10 subjects felt the spreading well

TABLE 8
Selected water-in-oil SPF Tinted moisturizer compositions

| | | Control | Example | Example | Example | Example |
|---|---|---|---|---|---|---|
| Material name En | INCI Name | 18 | 41 | 42 | 43 | 44 |
| DEIONIZED WATER | WATER | To 100 | To 100 | To 100 | To 100 | To 100 |
| VEGELIGHT C912-LC (GRANT | C9-12 ALKANE, COCO- | 5 | 5 | 5 | 10 | 5 |
| INDUSTRIES) | CAPRYLATE/CAPRATE | | | | | |
| NEOSSANCE SQUALANE | SQUALANE | 10 | 10 | 5 | 10 | 10 |
| BENTONE 38VCG | DISTEARDIMONIUM HECTORITE | 2 | 2 | 2 | 2.5 | 2.5 |
| ZEMEA | PROPANEDIOL | | 0.5 | 0.5 | 0.5 | 0.5 |
| WOGEL 18DV | Polyglyceryl-2 Diisostearate | 3 | 3 | 3 | 3 | 3 |
| ACZD SX | Isostearic Acid | 0.5 | 0.5 | 0.5 | 0.5 | 0.5 |
| SY - GLYSTERCRS - 75 | Polyglyceryl-6 Polyricinoleate | 1 | 1 | 1 | 1 | 1 |
| NEO HELIOPAN OS OCTISLTE | ETHYLHEXYL SALICYLATE | 5 | 5 | 5 | 5 | 5 |
| PARSOL 340 | Octocrylene | 5 | 5 | 5 | 5 | 5 |
| CRODAMOL OSU-LQ-(JP) | DIETHYLHEXYL SUCCINATE | 5 | 5 | 5 | 5 | 5 |
| ESP EcoCel 10 | EARTH SUPPLIED PRODUCTS | 2 | 2 | 2 | 2 | 2 |
| ASL-1 TIO2 CR-50 | Ci 77491 & Sodium Dilauramidoglutamide | 3 | 3 | 3 | 3 | 3 |
| | Lysine & Magnesium Chloride | | | | | |
| ASL-1 YELLOW LL-100P | Ci 77492 & Sodium Dilauramidoglutamide | 1.5 | 1.5 | 1.5 | 1.5 | 1.5 |
| | Lysine & Magnesium Chloride | | | | | |
| ASL-1 RED R-516P | Ci 77491 & Sodium Dilauramidoglutamide | 0.4 | 0.4 | 0.4 | 0.4 | 0.4 |
| | Lysine & Magnesium Chloride | | | | | |
| ASL-1 BLACK BL-100P | Ci 77499 & Sodium Dilauramidoglutamide | 0.1 | 0.1 | 0.1 | 0.1 | 0.1 |
| | Lysine & Magnesium Chloride | | | | | |
| TITANIUM DIOXIDE MT-100 TVS | TITANIUM DIOXIDE & ALUMINUM | 10 | 10 | 10 | 10 | 10 |
| | HYDROXIDE & STEARIC ACID | | | | | |
| STERLING USP SALT | SODIUM CHLORIDE | 0.2 | 0.2 | 0.2 | 0.2 | 0.2 |
| ZEMEA | PROPANEDIOL | 5 | 5 | 5 | 5 | 5 |
| GLYCERINE NAT 99.7% ACIDCHEM | GLYCERIN | 5 | 5 | 5 | 5 | 5 |
| 1,3 BUTYLENE GLYCOL | BUTYLENE GLYCOL | 4 | 4 | 4 | 4 | 4 |
| PHENOXETOL NF 272556 | PHENOXYETHANOL | 0.5 | 0.5 | 0.5 | 0.5 | 0.5 |
| (in water phase) | | | | | | |
| Evaluation | Viscosity (1 day) | 29200 | 29750 | 28200 | 21900 | 27000 |
| | Oil leaking (50 C. 4 weeks) | − | + | + | + | + |
| | Spreadability | + | + | + | + | + |

The data in Table 8 shows that In the W/O cream compositions, propanediol improved oil leaking. Propanediol worked well as the activator.

The compositions of Table 9 were prepared using the following Manufacturing Process: 1. Disperse Disteardiamonium Hectorite in oil phase with 3000 rpm 2. Add preservative bentone activator and disperse it with 3000 rpm 3. Add surfactant and disperse it with 3000 rpm 4. Add powder, inorganic UV filters, and pigments and disperse it with 4000 rpm. 5. Mix water phase and add it to oil phase with emulsification with 4000 rpm

The compositions of Table 9 were evaluated as follows

### Evaluation

### 1. Viscosity

Measured viscosity by BLOOKFIELD DV-1 Viscometer with LV-04, 12 rpm, 1 min

2. Oil leaking

Observed and evaluated the amount of oil leaking of samples at 50C for 4 weeks
+: No oil leaking
+/−: Tiny oil leaking
−: Apparent, noticeable oil leaking

### 3. Spreadability

+: More than 70% of 10 subjects felt spreading well
+/−: More than 40% of 10 subjects felt spreading well
−: Less than 40% of 10 subjects felt the spreading well

TABLE 9
Exemplary water-in-oil SPF foundation composition

| | | Control | Example | Example | Example | Example | Example |
|---|---|---|---|---|---|---|---|
| Material name En | INCI Name | 19 | 45 | 46 | 47 | 48 | 49 |
| DEIONIZED WATER | WATER | To 100 | To 100 | To 100 | To 100 | To 100 | To 100 |
| VEGELIGHT C912-LC (GRANT | C9-12 ALKANE, COCO- | 10 | 10 | 10 | 8 | 6 | 5 |
| INDUSTRIES) | CAPRYLATE/CAPRATE | | | | | | |
| NEOSSANCE SQUALANE | SQUALANE | 10 | 10 | 10 | 8 | 6 | 5 |
| BENTONE 38VCG | DISTEARDIMONIUM | 2.5 | 2.5 | 2.5 | 3 | 3.5 | 3.5 |
| | HECTORITE | | | | | | |
| ZEMEA | PROPANEDIOL | | 0.5 | | | | |
| PHENOXETOL NF 27255613162 | PHENOXYETHANOL | | | 0.5 | 0.7 | 0.7 | 0.7 |
| (in oil phase) | | | | | | | |
| WOGEL 18DV | Polyglyceryl-2 Diisostearate | 3 | 3 | 3 | 3 | 3 | 3 |
| ACZD SX | Isostearic Acid | 0.5 | 0.5 | 0.5 | 0.5 | 0.5 | 0.5 |
| SY - GLYSTERCRS - 75 | Polyglyceryl-6 Polyricinoleate | 1.2 | 1.2 | 1.2 | 1.2 | 1.2 | 1.2 |
| NEO HELIOPAN OS OCTISLTE | ETHYLHEXYL | 5 | 5 | 5 | 5 | 5 | 5 |
| | SALICYLATE | | | | | | |
| PARSOL 340 | Octocrylene | 5 | 5 | 5 | 5 | 5 | 5 |
| CRODAMOL OSU-LQ-(JP) | DIETHYLHEXYL | 5 | 5 | 5 | 5 | 5 | 5 |
| | SUCCINATE | | | | | | |
| MSS-500/N | Silica | 5 | 5 | 5 | 8 | 8 | 8 |
| ASL-1 TIO2 CR-50 | Ci 77491 & Sodium | 5 | 5 | 5 | 5 | 5 | 5 |
| | Dilauramidoglutamide Lysine | | | | | | |
| | & Magnesium Chloride | | | | | | |
| ASL-1 YELLOW LL-100P | Ci 77492 & Sodium | 3.2 | 3.2 | 3.2 | 3.2 | 3.2 | 3.2 |
| | Dilauramidoglutamide Lysine | | | | | | |
| | & Magnesium Chloride | | | | | | |
| ASL-1 RED R-516P | Ci 77491 & Sodium | 0.7 | 0.7 | 0.7 | 0.7 | 0.7 | 0.7 |
| | Dilauramidoglutamide Lysine | | | | | | |
| | & Magnesium Chloride | | | | | | |
| ASL-1 BLACK BL-100P | Ci 77499 & Sodium | 0.2 | 0.2 | 0.2 | 0.2 | 0.2 | 0.2 |
| | Dilauramidoglutamide Lysine | | | | | | |
| | & Magnesium Chloride | | | | | | |
| TITANIUM DIOXIDE MT-100 TVS | TITANIUM DIOXIDE & | 8 | 8 | 8 | 8 | 8 | 8 |
| | ALUMINUM HYDROXIDE | | | | | | |
| | & STEARIC ACID | | | | | | |
| STERLING USP SALT | SODIUM CHLORIDE | 0.2 | 0.2 | 0.2 | 0.2 | 0.2 | 0.2 |
| ZEMEA | PROPANEDIOL | 5 | 5 | 5 | 5 | 5 | 5 |
| GLYCERINE NAT 99.7% ACIDCHEM | GLYCERIN | 5 | 5 | 5 | 5 | 5 | 5 |
| 1,3 BUTYLENE GLYCOL | BUTYLENE GLYCOL | 4 | 4 | 4 | 4 | 4 | 4 |
| PHENOXETOL NF 272556 | PHENOXYETHANOL | 0.5 | 0.5 | 0.5 | 0.3 | 0.3 | 0.3 |
| (in water phase) | | | | | | | |
| Evaluation | Viscosity (1 day) | 17000 | 14450 | 15150 | 23900 | 33950 | 30950 |
| | Oil leaking (50 C. 4 weeks) | − | + | + | + | + | + |
| | Spreadability | + | + | + | + | + | + |

The data in Table 9 shows that phenoxyethanol and propanediol improved oil leaking. Increasing viscosity showed good stability and spreadability.

The compositions of Table 10 were prepared using the following Manufacturing Process: 1. Disperse Disteardiamonium Hectlite in oil phase with 3000 rpm; 2. Add preservative benton activator and disperse it with 3000 rpm; 3. Add serfactant and disperse it with 3000 rpm; 4. Add powder and inorganic UV filters, and disperse it with 4000 rpm; 5.Mix water phase and add it to oil phase with emulsification with 4000 rpm

The compositions of Table 10 were evaluated as follows:

### Evaluation

### 1. Viscosity

Measured viscosity by BLOOKFIELD DV-1 Viscometer with LV-04, 12 rpm, 1 min

2. Oil leaking

Observed and evaluated the amount of oil leaking of samples at 50C for 4 weeks
+: No oil leaking
+/−: Tiny oil leaking
−: Apparent, noticeable oil leaking

### 3. Spreadability

+: More than 70% of 10 subjects felt spreading well
+/−: More than 40% of 10 subjects felt spreading well
−: Less than 40% of 10 subjects felt the spreading well

TABLE 10
Exemplary water-in-oil SPF primer compositions

| Trade name | | Example 50 | Example 51 |
|---|---|---|---|
| DEIONIZED WATER | AQUA | To 100 | To 100 |
| VEGELIGHT C912-LC (GRANT INDUSTRIES) | C9-12 ALKANE, COCO-CAPRYLATE/CAPRATE | 5 | 6 |
| NEOSSANCE SQUALANE | SQUALANE | 5 | 8 |
| BENTONE 38VCG | Disteardimonium Hectorite | 2.5 | 1.5 |
| PHENOXETOL NF 27255613162 (in oil phase) | PHENOXYETHANOL | 0.5 | 0.5 |
| WOGEL 18DV | POLYGLYCERYL-2 DIISOSTEARATE | 4 | 4 |
| SY - GLYSTERCRS - 75 | Polyglyceryl-6 Polyricinoleate | 1 | 1 |
| ISOSTEARIC ACID SX | ISOSTEARIC ACID | 1 | 1 |
| ESTEMOL 182V | SORBITAN SESQUIISOSTEARATE | 2 | 2 |
| COCONAD RK (IWASE COSFA. CO., LTD) | TRICAPRYLIN | 10 | 10 |
| JOJOBA OIL | Simmondsia Chinensis Seed Oil | 4 | 6 |
| TITANIUM DIOXIDE MT-100 TV | Titanium Dioxide & Aluminum Hydroxide & Stearic | 8 | 8 |
| | Acid | | |
| MZX-304OTS | ZINC OXIDE & TRIETHOXYCAPRYL YLSILANE | 12 | 12 |
| SATINIER M5 (IKEDA CORPORATION OF AMERICA) | SILICA | 5 | 5 |
| EDTA 3.NA2.H20 | Trisodium edta | 0.2 | 0.2 |
| STERLING USP SALT | SODIUM CHLORIDE | 0.1 | 0.1 |
| GLYCERINE NAT 99.7% ACIDCHEM | GLYCERIN | 5 | 5 |
| ZEMEA | PROPANEDIOL | 8 | 8 |
| PHENOXETOL NF 272556 (in water phase) | PHENOXYETHANOL | 0.5 | 0.5 |
| Evaluation | Viscosity (1 day) | 14450 | 15150 |
| | Oil leaking (50 C 4 weeks) | + | + |
| | Spreadability | + | + |

The data in Table 10 shows that this phenoxyethanol activated oil gel, and achieved stable SPF primer compositions with good spreadability.

TABLE 11
Exemplary water-in-oil concealer compositions

| | | Example | Example | Example |
|---|---|---|---|---|
| Material name En | INCI Name | 51 | 52 | 53 |
| DEIONIZED WATER | WATER | To 100 | To 100 | To 100 |
| VEGELIGHT C912-LC (GRANT INDUSTRIES) | C9-12 ALKANE, COCO- | 4 | 4 | 4 |
| | CAPRYLATE/CAPRATE | | | |
| NEOSSANCE SQUALANE | SQUALANE | 8 | 8 | 8 |
| BENTONE 38VCG | DISTEARDIMONIUM | 2.5 | 2.5 | 2.5 |
| | HECTORITE | | | |
| LEXGARD E | Ethylhexylglycerin | 0.5 | 0.5 | 0.5 |
| WOGEL 18DV | Polyglyceryl-2 | 5 | 5 | 5 |
| | Diisostearate | | | |
| ACZD SX | Isostearic Acid | 1 | 1 | 1 |
| SY - GLYSTERCRS - 75 | Polyglyceryl-6 | 2 | 2 | 2 |
| | Polyricinoleate | | | |
| CARESS ® BN09 | Boron Nitride | 3 | 3 | 3 |
| TAYCA MP-25 MG | TITANIUM DIOXIDE & | 10 | 14 | 12 |
| | MAGNESIUM | | | |
| | STEARATE | | | |
| UNIPURE YELLOW LC 182 LL | IRON OXIDES (duplicate | 8 | 5 | 7 |
| | 2) & LAUROYL LYSINE | | | |
| UNIPURE RED LC 381 LL | IRON OXIDES (duplicate | 3 | 2 | 4 |
| | 1) & LAUROYL LYSINE | | | |
| UNIPURE BLACK LC 989 LL | IRON OXIDES (duplicate | 1 | 0.5 | 0.7 |
| | 3) & LAUROYL LYSINE | | | |
| TITANIUM DIOXIDE MT-100 TVS | TITANIUM DIOXIDE & | 8 | 0.5 | 0.5 |
| | ALUMINUM | | | |
| | HYDROXIDE & | | | |
| | STEARIC ACID | | | |
| STERLING USP SALT | SODIUM CHLORIDE | 0.2 | 0.2 | 0.2 |
| ZEMEA | PROPANEDIOL | 5 | 5 | 5 |
| GLYCERINE NAT 99.7% ACIDCHEM | GLYCERIN | 5 | 5 | 5 |
| 1,3 BUTYLENE GLYCOL | BUTYLENE GLYCOL | 4 | 4 | 4 |
| PHENOXETOL NF 272556 (in water phase) | PHENOXYETHANOL | 0.5 | 0.5 | 0.5 |

The compositions of Table 11 were prepared using the following manufacturing process: 1. Disperse Disteardiamonium Hectlite in oil phase with 3000 rpm; 2. Add preservative benton activator and disperse it with 3000 rpm; 3. Add serfactant and disperse it with 3000 rpm; 4. Add powder and inorganic UV filters, and disperse it with 4000 rpm; 5. Mix water phase and add it to oil phase with emulsification with 4000 rpm.

TABLE 12
Exemplary water-in-oil skincare cream compositions

| | | Example | Example | Example |
|---|---|---|---|---|
| Material name En | INCI Name | 54 | 55 | 56 |
| DEIONIZED WATER | WATER | To 100 | To 100 | To 100 |
| VEGELIGHT C912-LC (GRANT INDUSTRIES) | C9-12 ALKANE, COCO- | 5 | 5 | 5 |
| | CAPRYLATE/CAPRATE | | | |
| NEOSSANCE SQUALANE | SQUALANE | 20 | 20 | 20 |
| BENTONE 38VCG | DISTEARDIMONIUM | 3 | 3 | 3 |
| | HECTORITE | | | |
| Zeastat ™ | CaprylhydroxamicAcid | 1 | | |
| | and Propanediol | | | |
| Lexgard ™Natural MHG MB | Methylheptylglycerin | | 1 | |
| BRONTIDE (genomatica) | Butylene Glycol | | | 1 |
| WOGEL 18DV | Polyglyceryl-2 | 4 | 4 | 4 |
| | Diisostearate | | | |
| ACZD SX | Isostearic Acid | 0.5 | 0.5 | 0.5 |
| SY - GLYSTERCRS - 75 | Polyglyceryl-6 | 2 | 2 | 1 |
| | Polyricinoleate | | | |
| JOJOBA OIL | Simmondsia Chinensis | 5 | 5 | 5 |
| | Seed Oil | | | |
| BOTANESSENTIAL OLIVE S-EHO | ETHYLHEXYL | 5 | 5 | 5 |
| | OLIVATE, SQUALANE | | | |
| CRODAMOL OSU-LQ-(JP) | DIETHYLHEXYL | 5 | 5 | 5 |
| | SUCCINATE | | | |
| RAIN FOREST 03410 (REFINED CUPUA [symbol] U | THEOBROMA | 3 | 5 | 5 |
| BUTTER) | GRANDIFLORUM | | | |
| | SEED BUTTER, | | | |
| | TOCOPHEROL | | | |
| STERLING USP SALT | SODIUM CHLORIDE | 0.2 | 0.2 | 0.2 |
| ZEMEA (in water phase) | PROPANEDIOL | 10 | 10 | 10 |
| GLYCERINE NAT 99.7% ACIDCHEM | GLYCERIN | 5 | 5 | 5 |
| PHENOXETOL NF 272556 (in water phase) | PHENOXYETHANOL | 0.4 | 0.4 | 0.4 |

The compositions of Table 12 were prepared using the following manufacturing process: 1. Disperse Disteardiamonium Hectlite in oil phase with 3000 rpm; 2. Add preservative benton activator and disperse it with 3000 rpm; 3. Add serfactant and disperse it with 3000 rpm; 4. Add powder and inorganic UV filters, and disperse it with 4000 rpm; 5. Mix water phase and add it to oil phase with emulsification with 4000 rpm.

TABLE 13
Exemplary water-in-oil SPF 30 Tinted Moisturizer with a Non Silicone solvent

| Trade name | | Example 57 | Example 58 |
|---|---|---|---|
| DEIONIZED | AQUA | To 100 | To 100 |
| WATER | | | |
| VEGELIGHT C912- | C9-12 ALKANE, COCO- | 10 | 10 |
| LC (GRANT | CAPRYLATE/CAPRATE | | |
| INDUSTRIES) | | | |
| PARLEAM 3 | HYDROGENATED POLYISOBUTENE, | 4.7 | 4.2 |
| | TOCOPHEROL | | |
| NEOSSANCE | SQUALANE | 0.5 | 1 |
| SQUALANE | | | |
| DERMOL 99 | ISONONYL ISONONANOATE | 2 | 2 |
| BENTONE 38VCG | Disteardimonium Hectorite | 2 | 2 |
| PHENOXETOL NF | PHENOXYETHANOL | 0.5 | 0.5 |
| 27255613162 (in oil | | | |
| phase) | | | |
| WOGEL 18DV | POLYGLYCERYL-2 DIISOSTEARATE | 1 | 1 |
| SY - GLYSTERCRS - | Polyglyceryl-6 Polyricinoleate | 3 | 3 |
| 75 | | | |
| ISOSTEARIC ACID | ISOSTEARIC ACID | 0.5 | 0.5 |
| SX | | | |
| PARSOL ® 340 | OCTOCRYLENE | 5 | 5 |
| CRODAMOL OSU- | DIETHYLHEXYL SUCCINATE | 5 | 5 |
| LQ-(JP) | | | |
| NEO HELIOPAN ® | ETHYLHEXYL SALICYLATE | 4.8 | 4.8 |
| OS | | | |
| TITANIUM | Titanium Dioxide & Aluminum Hydroxide | 8 | 12 |
| DIOXIDE MT-100 | & Stearic Acid | | |
| TV | | | |
| NHS-TR-10 | TITANIUM DIOXIDE, ALUMINUM | 2.62 | 2.62 |
| | HYDROXIDE, ISOSTEARYL | | |
| | SEBACATE, DISODIUM STEAROYL | | |
| | GLUTAMATE | | |
| NHS-YP-10 | IRON OXIDES, ISOSTEARYL | 1.45 | 1.45 |
| | SEBACATE, DISODIUM STEAROYL | | |
| | GLUTAMATE, ALUMINUM | | |
| | HYDROXIDE | | |
| NHS-RPS-10 | IRON OXIDES, ISOSTEARYL | 0.26 | 0.26 |
| | SEBACATE, DISODIUM STEAROYL | | |
| | GLUTAMATE, ALUMINUM | | |
| | HYDROXIDE | | |
| NHS-BP-10 | IRON OXIDES, ISOSTEARYL | 0.17 | 0.17 |
| | SEBACATE, DISODIUM STEAROYL | | |
| | GLUTAMATE, ALUMINUM | | |
| | HYDROXIDE | | |
| CHIFFONSIL P-3R | SILICA | 7 | 7 |
| ZEMEA | PROPANEDIOL | 5 | 5 |
| GLYCERINE NAT | GLYCERIN | 5 | 5 |
| 99.7% ACIDCHEM | | | |
| 1,3 BUTYLENE | BUTYLENE GLYCOL | 4 | 4 |
| GLYCOL | | | |
| PHENOXETOL NF | PHENOXYETHANOL | 0.4 | 0.4 |
| 272556 (in water | | | |
| phase) | | | |
| SUPERIOR TX-10 | SODIUM CHLORIDE | 1 | 1 |
| SALT | | | |
| Evaluation | Viscosity (1 day) | 17,420 | 16,580 |
| | Oil leaking (50 C 4 weeks) | + | + |
| | Spreadability | + | + |

The compositions of Table 13 was prepared and evaluated using methods, which are similar to those for preparing and evaluating the compositions of Tables 1-12.

Table 13 show that Phenoxyethanol activated the oil phase, and achieved stable SPF with good spreadability no oil leaking.

TABLE 14
Exemplary water-in-oil SPF 30 Tinted Moisturizer with a Silicone solvent.

| | | Example | Example |
|---|---|---|---|
| Trade name | | 59 | 60 |
| DEIONIZED | AQUA | To 100 | To 100 |
| WATER | | | |
| CRODAMOL | DIETHYLHEXYL SUCCINATE | 5 | 5 |
| OSU-LQ-(JP) | | | |
| NEO | ETHYLHEXYL SALICYLATE | 4.8 | 4.8 |
| HELIOPAN ® OS | | | |
| PARSOL ® 340 | OCTOCRYLENE | 5 | 5 |
| WOGEL 18DV | POLYGLYCERYL-2 DIISOSTEARATE | 3 | 3 |
| ISOSTEARIC | ISOSTEARIC ACID | 1 | 1 |
| ACID EX | | | |
| BENTONE | DISTEARDIMONIUM HECTORITE | 2 | 2 |
| 38VCG | | | |
| PHENOXETOL | PHENOXYETHANOL | 0.5 | 0.5 |
| NF 272556 | | | |
| DM-FLUID-2CS | DIMETHICONE | 12 | 13 |
| (KF-96A-2CS) | | | |
| BELSIL ® DM 1 | DIMETHICONE | 8 | 8 |
| PLUS | | | |
| CHIFFONSIL P- | SILICA | 5 | 5.5 |
| 3R | | | |
| TITANIUM | TITANIUM DIOXIDE, ALUMINUM | 8 | 8 |
| DIOXIDE MT- | HYDROXIDE, STEARIC ACID | | |
| 100 TVS | | | |
| ASL-1 TIO2 CR- | TITANIUM DIOXIDE, ALUMINUM | 2.7 | 2.8 |
| 50 | HYDROXIDE, SODIUM | | |
| | DILAURAMIDOGLUTAMIDE LYSINE, | | |
| | MAGNESIUM CHLORIDE | | |
| ASL-1 YELLOW | IRON OXIDES, SODIUM | 1.45 | 1.32 |
| LL-100P | DILAURAMIDOGLUTAMIDE LYSINE, | | |
| | MAGNESIUM CHLORIDE | | |
| ASL-1 RED R- | IRON OXIDES, SODIUM | 0.2 | 0.22 |
| 516P | DILAURAMIDOGLUTAMIDE LYSINE, | | |
| | MAGNESIUM CHLORIDE | | |
| ASL-1 BLACK | IRON OXIDES, SODIUM | 0.15 | 0.16 |
| BL-100P | DILAURAMIDOGLUTAMIDE LYSINE, | | |
| | MAGNESIUM CHLORIDE | | |
| ZEMEA | PROPANEDIOL | 5 | 5 |
| GLYCERINE | GLYCERIN | 5 | 5 |
| NAT 99.7% | | | |
| ACIDCHEM | | | |
| 1,3 BUTYLENE | BUTYLENE GLYCOL | 4 | 4 |
| GLYCOL | | | |
| PHENOXETOL | PHENOXYETHANOL | 0.4 | 0.4 |
| NF 272556 (in | | | |
| water phase) | | | |
| SUPERIOR TX- | SODIUM CHLORIDE | 0.2 | 0.2 |
| 10 SALT | | | |
| Evaluation | Viscosity (1 day) | 44,580 | 25,250 |
| | Oil leaking (50 C 4 weeks) | + | + |
| | Spreadability | + | + |

The compositions of Table 14 were prepared and evaluated using methods, which are similar to those for preparing and evaluating the compositions of Tables 1-12.

Table 14 shows that Phenoxyethanol activated the oil phase, and achieved stable SPF with good spreadability no oil leaking.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

1. A cosmetic composition comprising:
(a) a modified hectorite organoclay;
(b) at least one hydrophobic solvent; and
(c) at least one amphiphilic agent.

2. The cosmetic composition of claim 1, wherein the composition is a water-in-oil type emulsified composition.

3. The cosmetic composition of claim 1, wherein the modified hectorite organoclay is disteardimonium hectorite.

4. The cosmetic composition of claim 1, wherein an amount of the modified hectorite organoclay in the composition is from 0.01 mass % to 20 mass %.

5. The cosmetic composition of claim 1, wherein the at least one hydrophobic solvent comprises at least one oil solvent.

6. The cosmetic composition of claim 5, wherein the at least one oil solvent comprises at least one hydrocarbon oil.

7. The cosmetic composition of claim 5, wherein the at least one oil solvent comprises at least one non-polar oil solvent.

8. The cosmetic composition of claim 5, wherein the at least one oil solvent comprises a volatile oil and a non-volatile oil.

9. The composition of claim 8, wherein a mass ratio between the volatile oil and the non-volatile oil is from 10:1 to 1:10

10. The composition of claim 1, wherein the at least one amphiphilic agent comprises at least one liquid preservative.

11. The composition of claim 1, wherein the at least one amphiphilic agent comprises at least one ingredient selected from diols, aromatic alcohols, glycerin ethers and combinations thereof.

12. The composition of claim 1, wherein the at least one amphiphilic agent comprises at least one ingredient selected from the group consisting of phenoxyethanol, methylheptylglycerin, ethylhexylglycerin, caprylhydroxamic acid, propanediol, butylene glycol and combinations thereof.

13. The composition of claim 1, wherein an amount of the at least one amphiphilic agent is from 0.05 mass % to 20.0 mass % of the composition.

14. The composition of claim 1 further comprising at least one amphiphilic surfactant.

15. The composition of claim 14, wherein the at least one amphiphilic surfactant is at least one non-PEG surfactant.

16. The composition of claim 14, wherein the at least one amphiphilic surfactant comprises at least one polyglyceryl surfactant.

17. The composition of claim 14, wherein the at least one amphiphilic surfactant comprises at least one surfactant selected from polyglyceryl-2 diisostearate, polyglyceryl-6 polyricinoleate, sorbitan sesquiisostearate and combinations thereof.

18. The composition of claim 14, wherein an amount of the at least one amphiphilic surfactant is from 0.1 mass % to 15 mass % of the composition.

19. The composition of claim 1, further comprising one or more UV scattering powder.

20. The composition of claim 1, further comprising one or more UV absorbing organic molecules.

21. The composition of claim 1, further comprising one or more pigments.

22. The composition of claim 1, which is free of a PEG ingredient.

23. The composition of claim 1, which is free of microplastic particles.

24. The composition of claim 1 comprising:
0.5 mass % to 5 mass % of disteardimonium hectorite;
5 mass % to 15 mass % of a volatile oil solvent;
5 mass % to 15 mass % of a non-volatile oil solvent;
0.1 mass % to 10.0 mass % of at least one ingredient selected from the group consisting of phenoxyethanol, methylheptylglycerin, ethylhexylglycerin caprylhydroxamic acid, propanediol, butylene glycol and combinations thereof, and
1 mass % to 7 mass % of at least one surfactant selected from polyglyceryl-2 diisostearate, polyglyceryl-6 polyricinoleate, and a combination thereof.

25. A cosmetic method comprising applying the cosmetic composition of claim 1 to a keratinous surface.
